(19) United States
(12) Patent Application Publication
(10) Pub. No.: US 2012/0052004 A1
(43) Pub. Date: Mar. 1, 2012

(54) CARBON NANOTUBE BASED MAGNETIC RESONANCE IMAGE CONTRAST AGENTS

(76) Inventors: Lon J. Wilson, Houston, TX (US); Jeyarama Subramanian Ananta Narayanan, Houston, TX (US); Balaji Sitharaman, Coram, NY (US); Keith Bennett Hartman, McLean, VA (US)

(21) Appl. No.: 13/022,286

(22) Filed: Feb. 7, 2011

Related U.S. Application Data
(63) Continuation-in-part of application No. PCT/US2009/053274, filed on Aug. 10, 2009.
(60) Provisional application No. 61/087,198, filed on Aug. 8, 2008.

(51) Int. Cl. A61K 51/00; A61P 43/00; A61K 49/00; A61K 49/06; B82Y 5/00

(52) U.S. Cl. 424/1.11; 424/9.1; 424/9.36; 977/773; 977/930

(57) ABSTRACT

A contrast agent composition comprising at least one carbon nanotube and a metal catalyst. A method for obtaining a magnetic resonance image, the method comprising: administering to a subject a contrast agent composition, wherein a contrast agent composition comprises at least one carbon nanotube and a metal catalyst; and obtaining a magnetic resonance image of at least a portion of the subject in which the contrast agent is disposed.

### CROSS-REFERENCE TO RELATED APPLICATIONS

This application is a continuation-in-part of International Application No. PCT/US2009/053274, filed Aug. 10, 2009, which claims the benefit of U.S. Provisional Patent Application Ser. No. 61/087,198, filed Aug. 8, 2008, the entire disclosures of which are hereby incorporated by reference.

### STATEMENT OF GOVERNMENT INTEREST

The present invention was made with support under Grant Number EEC-0647452 awarded by the National Science Foundation. The U.S. government has certain rights in the invention.

### BACKGROUND

The present invention relates generally to contrast agent compositions. In particular, the present invention relates to compositions of carbon nanotube based contrast agents and associated methods of use.

Contrast agents (CAs) play a prominent role in magnetic resonance imaging (MRI) in medicine. MRI CAs are primarily used to improve disease detection by increasing sensitivity and diagnostic confidence. There are several types of MR contrast agents being used in clinical practice today. These include extracellular fluid space (ECF) agents, extended residence intravascular blood pool agents, and tissue(organ)-specific agents Annually, approximately sixty million MRI procedures are performed worldwide and around 30% of these procedures use MRI CAs. The lanthanide ion, Gd³⁺, is usually chosen for MRI CAs because it has a very large magnetic moment (μ²=63 μB²) and a symmetric electronic ground state, ⁸S_{7/2}. The aquated Gd³⁺ ion is toxic and hence is sequestered by chelation or encapsulation in order to reduce toxicity. However, in vivo release of such metal ions can occur. Gd³⁺-metal chelate-based agents have been shown to cause nephrogenic systemic fibrosis (NSF) in patients with renal dysfunction.

MRI CAs are generally used to improve sensitivity and diagnostic confidence, and they are classified into two types: 1) spin-lattice relaxation agents [T₁-shortening agents like paramagnetic Gd³⁺, Mn²⁺, etc.] or 2) spin-spin relaxation agents [T₂-shortening agents like superparamagnetic iron oxide (SPIO) nanoparticles] where T_{1,2} are the proton relaxation times.

Since their discovery in 1991, carbon nanotubes have found wide-spread potential for various technological applications. In particular, their hollow interior coupled with a chemically-modifiable outer surface makes them intriguing candidates as diagnostic and therapeutic agents in medicine. Single-walled carbon nanotubes (SWNTs), which can be described as hollow cylinders made from single sheets of graphene, are among the most investigated form of carbon nanotubes for biological and medical applications. The ideal SWNT length for biological applications is still unknown, however, ultra-short SWNTs (US-tubes), 20-100 nm in length, might be especially good candidates for such applications. Such US-tubes have already been shown to be high-performance T₁-weighted MRI contrast agents when internally loaded with Gd³⁺ ions, X-ray contrast agents when internally filled with molecular iodine (I₂), and α-radiotherapeutic agents when internally doped with AtCl molecules.

While the present disclosure is susceptible to various modifications and alternative forms, specific example embodiments are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DRAWINGS

FIG. 1 shows T₂-weighted MRI phantom images of the SWNT samples in a 3 T scanner at different echo times.

FIG. 2 shows Powder X-ray Diffraction pattern of the SWNT materials.

FIG. 3 shows Zero-field-cooled [black] and field-cooled [white] magnetization curves for a) r-SWNTs b) p-SWNTs c) US-tubes. Applied magnetic field is 0.1 T.

FIG. 4 shows a microscopic image of gadonanotubes labeled J774 cells.

FIG. 5 shows ΔR₂ and ΔR₂* relaxation rates at increasing number of gadonanotubes labeled cells in 1.0% agarose gel at 3 T.

FIG. 6 shows T₂*-weighted images of gadonanotubes labeled J774 cells at 9.4 T, using SWI reconstruction.

### DESCRIPTION

The present invention relates generally to contrast agent compositions. In particular, the present invention relates to compositions of carbon nanotube based contrast agents and associated methods of use.

The present disclosure provides, in certain embodiments, a contrast agent composition comprising at least one carbon nanotube and a metal catalyst.

The present disclosure provides, in certain embodiments, a contrast agent composition consisting essentially of at least one carbon nanotube and a metal catalyst.

The compositions of the present invention exhibit a number of advantageous characteristics. Such characteristics include, but are not limited to, very strong T₂-relaxation (spin-spin relaxation or transverse relaxation) and very high relaxivity (efficiency of an agent to reduce the water proton relaxation time and to act as a contrast agent in MRI scans) compared to the commercially-available T₂-weighted clinical contrast agents.

The carbon nanotubes useful in the compositions and methods of the present invention may be any suitable carbon nanotube. In certain embodiments, single-walled carbon nanotubes (SWNTs) may be useful in the compositions and methods of the present invention. SWNTs possess unique characteristics that make them desirable for biomedical applications. SWNTs, also known as single walled tubular fullerenes, are cylindrical molecules consisting essentially of sp² hybridized carbons. In defining the size and conformation of single-walled carbon nanotubes, the system of nomenclature described by Dresselhaus et al., Science of Fullerenes and Carbon Nanotubes, Ch. 19, ibid. will be used. Single walled tubular fullerenes are distinguished from each other by a double index (x,y), where x and y are integers that describe how to cut a single strip of hexagonal graphite such that its edges join seamlessly when the strip is wrapped onto the surface of a cylinder. When x=y, the resultant tube is said to be of the “arm-chair” or (x,x) type, since when the tube is cut perpendicularly to the tube axis, only the sides of the hexagons are exposed and their pattern around the periphery of the tube edge resembles the arm and seat of an arm chair repeated n times. When y=0, the resultant tube is said to be of the “zig-zag” or (x,0) type, since when the tube is cut perpendicular to the tube axis, the edge is a zig-zag pattern. Where x≠y and y≠0, the resulting tube has chirality. The electronic properties of the nanotube are dependent on, among other things, the conformation. For example, arm-chair tubes are metallic and have, among other things, extremely high electrical conductivity. Other tube types may be metallic, semi-metals or semi-conductors, depending on their conformation. Regardless of tube type, all SWNTs may have, among other things, extremely high thermal conductivity and tensile strength. In certain embodiments, the SWNT may be a cylinder with two open ends, a cylinder with one closed end, or a cylinder with two closed ends. In certain embodiments, an end of an SWNT may be closed by a hemifullerene, for example a (10,10) carbon nanotube can be closed by a 30-carbon hemifullerene. If the SWNT has one or two open ends, the open ends may have any valences unfilled by carbon-carbon bonds within the single wall carbon nanotube filled by bonds with hydrogen, hydroxyl groups, carboxyl groups, or other groups. SWNTs may also be cut into ultra-short pieces, thereby forming US-tubes.

In certain embodiments, ultra-short carbon nanotubes (US-tubes) may be useful in the compositions and methods of the present invention. As used herein, the term “US-tubes” refers to ultra short carbon nanotubes with lengths from about 20 nm to about 200 nm. US tubes may be prepared by cutting SWNTs into ultra-short lengths. In certain embodiments, the carbon nanotubes used in the compositions of the present disclosure may comprise US tubes of length in the range of about 20 nm to about 80 nm. In certain embodiments, the carbon nanotubes used in the compositions of the present disclosure may comprise US tubes of a length of less than 100 nm.

The ideal length for medical applications is uncertain, but US-tubes may be well suited for cellular uptake, biocompatibility, and eventual elimination from the body. Additionally, the US-tube exterior surface may provide a versatile scaffold for attachment of chemical groups for solubilizing or targeting purposes, while its interior space allows for encapsulation of atoms, ions, and even small molecules whose cytotoxicity may be sequestered within the short carbon nanotube. Finally, medical imaging agents derived from US-tubes hold promise for intracellular imaging, since carbon nanotubes have been shown to translocate into the interior of cells with minimal cytotoxicity.

The carbon nanotubles useful in the compositions and methods of the present invention may be produced by any means known to one of ordinary skill in the art. In certain embodiments, the carbon nanotubes useful in the compositions and methods of the present invention may be produced by electric arc discharge. In certain embodiments, the carbon nanotubes useful in the compositions and methods of the present invention may be produced by high pressure CO conversion (HiP_{CO}). A substantial amount previous research concerning the loading of SWNT samples has been performed with electric-arc discharge-produced SWNTs as opposed to other SWNT production methods, such as high-pressure carbon monoxide (HiP_{CO}).

This is because, in many cases, arc-produced SWNTs have, among other things, a larger diameter than HiP_{CO} SWNTs (1.4 nm average diameter for arc vs. 1.0 nm diameter for HiPco) and arc SWNTs may contain more sidewall defects than HiPco SWNTs, thereby facilitating loading. For medical applications, however, the uniformity and purity of HiP_{CO} SWNTs may advantageous. Suitable commercially available carbon nanotubes may be obtained from Carbon Nanotechnologies Inc., Houston, Tex.

In certain embodiments, such methods of producing US tubes may comprise cutting full-length SWNTs into short pieces by a four-step process. First, residual iron catalyst particles may be removed by oxidation via exposure to wet-air or SF₆ followed by a strong acid (HCl) treatment to extract the oxidized iron particles. The purified SWNTs may then be fluorinated by a gaseous mixture of 1% F₂ in He at elevated temperatures for up to 2 hours and cut into short pieces by pyrolysis under argon at 900° C. The fluorination reaction may produce F-SWNTs, with a stoichiometry of CFₓ (x<0.2), which may comprise bands of fluorinated-SWNT separated by regions of pristine SWNT. Pyrolysis under Ar, among other things, liberates volatile fluorocarbons, thereby cutting the SWNTs into pieces with lengths corresponding to the areas of pristine SWNT. While this method known in the art is effective at producing cut SWNTs, improvements can be made; for example, the separate purification step is unnecessary and can be eliminated. Such improvements, provided that they do not adversely affect the compositions and methods of the present invention, are considered within this spirit of the present invention.

In certain embodiments, a three-step process of producing US tubes may be used. First, as produced HiP_{CO} SWNTs may be fluorinated in a monel steel apparatus by a mixture of 1% F₂ in He at 100° C. for about 2 hours. During this process, both the SWNTs and the iron catalyst particles may become at least partially fluorinated. Subsequent exposure to concentrated HCl may substantially remove the fluorinated catalyst particles without affecting the F-SWNTs, which have a stoichiometry of ˜C₁₀F after the acid treatment. The now-purified F-SWNTs are cut into US tubes by pyrolysis under Ar at 900° C. In certain embodiments, the resulting US tubes have lengths ranging from 20-80 nm, with the majority being ˜40 nm in length. Utilizing this method, the amount of iron catalyst may be reduced from ˜25 mass percent in raw SWNTs to ˜1 mass percent for US tubes. Therefore, in certain embodiments, this method may be ideal for the purification of SWNTs, but only as a precursor to producing US tubes. This is because the fluorine remaining, after the HCl acid treatment, is difficult to remove, making the F-SWNTs only viable for subsequent cutting. Furthermore, the time to produce US tubes from SWNTs using this method may be significantly reduced.

The carbon nanotubes can be substituted or unsubstituted. By “substituted” it is meant that a group of one or more atoms is covalently linked to one or more atoms of the carbon nanotube. In certain embodiments, Bingel chemistry may be used to substitute the nanotube with appropriate groups. Examples of groups suitable for use in the compositions and methods of the present invention may include, but are not limited to, malonate groups, serinol malonates, groups derived from malonates, serinol groups, serinol amide, carboxylic acid, dicarboxylic acid, polyethyleneglycol (PEG), t-butylphenylene groups, and the like. The synthesis of substituted carbon nanotubes is described in further detail in X. Shi, J. L. Hudson, P. P. Spicer, J. M. Tour, R. Krishnamoorti, A. G. Mikos, Biomacromolecules 7, 2237-2242 (2006), the entire disclosure of which is incorporated by reference to the extent it provides information available to one of skill in the art regarding the implementation of the technical teachings of the present invention.

The metal catalysts present in the compositions of the present invention may be any metal catalyst used in the catalytic growth process to create the carbon nanotube. Suitable metal catalysts may include, but are not limited to, Fe, Fe₂O₃, Y/Ni, and Y₂O₃/NiO. In certain embodiments, Fe or Fe₂O₃ may be present in the compositions and methods of the present invention when the carbon nanotubes are produced by HiP_{CO}. In certain embodiments, Y/Ni or Y₂O₃/NiO may be present in the compositions and methods of the present invention when the carbon nanotubes are produced by electric arc discharge. In certain embodiments, the metal catalyst may be present in the compositions of the present invention in an amount of less than about 10% by weight of the composition. In certain embodiments, the metal catalyst may be present in the compositions of the present invention in an amount of less than about 5% by weight of the composition. In certain embodiments, the metal catalyst may be present in the compositions of the present invention in an amount of less than about 2% by weight of the composition. In certain embodiments, the metal catalyst may be present in the compositions of the present invention in an amount of from about 0.5 to about 2% by weight of the composition.

In certain embodiments, the metal catalyst may be present in the compositions of the present invention is such an amount that it may not be removed from the compositions by conventional techniques. For example, in certain embodiments, the metal catalyst may be present in the compositions of the present invention in an amount which cannot be removed by one or more of the following techniques: oxidation by F₂ gas, pyrolysis at 1000° C., and washing with concentrated HCl. Such amounts of metal catalyst may be suitable because, among other things, removing such amounts would require extensive, and potentially expensive, procedures which may damage or alter the carbon nanotubes. Furthermore, such amounts may be suitable because, if one or more of the above-listed methods cannot remove the metal catalyst, little or no significant in vivo release of the metal catalyst from the carbon nanotube may occur.

Other suitable materials may be added to the compositions of the present invention. For example, the presence of the hollow interior of the carbon nanotube may allow materials including, but not limited to, multi-modal imaging agents and drugs to be administered by being contained substantially within the interior of the carbon nanotube. The exterior wall of carbon nanotube may also allow for the attachment of multi-modal imaging agents, targeting agents (including, but not limited to, peptides and antibodies) and/or therapeutic agents (including, but not limited to, chemotherapeutic agents and radiotherapeutic agents).

In certain embodiments, a SWNT may be used as carrier for efficient delivery of biomolecules such as drugs and genes into targeting sites for therapeutic purposes. In order to monitor the delivery location and efficiency, the SWNT may be loaded with gadolinium. Gadolinium loaded ultra-short single-walled nanotubes (gadonanotubes) can be used as high relaxivities r₁ and r₂ agents and can be functionalized for targeted delivery. The intracellular uptake of gadonanotubes exhibits a linear change of transverse relaxivities (R₂ and R₂*) with concentration with R₂* being the dominant relaxation mechanism at 3 T. Monitoring drug delivery dose encapsulated in the gadonanotubes can be done by quantification of R₂*. Single cell visualization of gadonanotubes is possible with high performance gradient to achieve 50 μm isotropic resolution, which is not possible with the gradient performance on clinical system.

Although gadonanotubes may not be as effective as a molecular contrast agent alone compared to iron oxide, the possibility of using gadonanotubes as drug and gene delivery carriers has immense potential to visualize the molecular imaging target and quantify the amount of drug and gene biomolecules being delivered. To facilitate a better understanding of the present invention, the following examples of specific embodiments are given. In no way should the following examples be read to limit or define the entire scope of the invention.

### EXAMPLES

### Example 1

Full-length SWNTs were fluorinated with 1% gaseous F₂ diluted with helium to yield partially-fluorinated nanotubes (fluoronanotubes). As produced, fluoronanotubes were then pyrolysed at 1000° C. under argon atmosphere to yield US-tubes. As obtained, US-tubes were sonicated with concentrated hydrochloric acid for 30 minutes to remove metal impurities and washed with several aliquots of de-ionized (DI) water and then dried overnight at 60° C.

Part of the dried US-tube sample was then loaded with Gd³⁺ by soaking and sonicating them in HPLC grade DI water (pH 5 7) containing aqueous GdCl₃.

The US-tubes, the parent SWNTs and the gadonanotubes were dispersed in bio-compatible pluronic F-108 surfactant for the relaxivity study results in Table 1.

TABLE 1

| | T₂ (ms) | |
|---|---|---|
| | per mg of | |
| Sample | nanotube | Metal weight % |
| US-tubes (made from full-length | 4.6 | Nickel 5.33% |
| SWNTs produced by electric arc | | Yttrium 3.40% |
| discharge) | | |
| US-tubes | 40.7 | Iron 0.63% |
| (made from HiP_{CO} SWNTs) | | |
| Full-length single-walled carbon | 0.9 | Yttrium 5.93% |
| nanotubes (produced by electric | | Nickel 24.37% |
| arc discharge method) | | |
| Full-length single-walled carbon | 2.9 | Iron 17.15% |
| nanotubes (produced by HiP_{CO} | | |
| process) | | |
| Gadonanotubes (made from arc- | 3.8 | Gadolinium 4.29% |
| produced US-tubes) | | Nickel 2.7% |
| | | Yttrium 0.63% |

### Example 2

In this example, we report that raw SWNTs (r-SWNTs), purified SWNTs (p-SWNTs) and US-tubes are also inherently high-performance T₂-weighted MRI contrast agents by virtue of their superparamagnetic character, with the US tubes being the most efficacious of the materials. The r-SWNTs were produced by the HiP_{CO} process (Carbon Nanotechnologies, Inc). As obtained, the r-SWNTs (˜17% iron catalyst) were then purified using a liquid bromine (Br₂) protocol that efficiently removes the iron catalyst impurities without significant nanotube sidewall damage to produce p-SWNTs (˜6% iron). The p-SWNTs were cut into ultrashort SWNTs (US-tubes) by fluorination and pyrolysis in an inert atmosphere. The cutting process produces nanocapsules with lengths predominantly between 20-100 nm, with significant damage to the nanotube sidewalls; metal ions and small molecules can be internally loaded through these sidewall defect sites. The three SWNT materials studied were dispersed in equal volumes of bio-compatible Pluronic® (polyethylene oxide-polypropylene oxide block co-polymer) surfactant for the MRI studies. The iron content of each SWNT sample was determined using inductively-coupled plasma-optical emission spectrometry (ICP-OES, Perkin-Elmer Optima 3200V). Magnetic properties of the samples were characterized with a Quantum Design MPMS-XL magnetometer based on a superconducting quantum interference device (SQUID) in the temperature range 5-300 K with an applied magnetic field of 0.1 T. Samples were encapsulated in diamagnetic cellulose for measurements and run in duplicate. X-ray powder diffraction (XRD) data were obtained using a RigakuD/Max-2100PC diffrractometer operating with unfiltered Copper Kα radiation (λ=1.5406 Å) at 40 kV and 40 mA. The contribution from the Kα2 radiation was compensated for using the Rachinger algorithm. Goniometer alignment was verified by daily analysis of a Rigaku-supplied SiO₂ reference standard. Processing of the powder diffraction results and phase identification was accomplished using the program JADE. The T₂-proton relaxation studies were performed on a 3 T MRI system (General electric, Milwaukee, Wis.), and the phantom images were obtained using 2D spin-echo imaging with a retention time (TR) of 500 ms and echo times (TEs) ranging from 10 to 50 ms in either 5 or 10 ms increments. For all T2-relaxation measurements, the samples were dispersed in Pluronic® surfactant, although the three SWNT materials disperse differently in surfactant. To normalize this effect, dilutions were made to produce equal quantities of the SWNT material in all three sample solutions studied. Initially, known quantities of each of the three materials in the absence of surfactant were analyzed for their iron content by ICP-OES. Three different ICP measurements (agreement within 2%) were used to determine the average iron content in each sample, as shown in Table 2. The SWNT samples were then dispersed in surfactant. The resulting suspensions were analyzed for iron content, and, from these values, the correct amount of SWNT sample was diluted such that the of SWNTs in each sample was the same (360±4 mg of SWNTs/L) as shown in Table 2. All the SWNT solutions had remarkably short T₂-relaxation

TABLE 2

| SWNT | Fe % | Fe concentration | T₂ time | r₂ relaxivity |
|---|---|---|---|---|
| Material | (% wt) | (mM) | (ms) | (mM⁻¹s⁻¹) |
| r-SWNTs | 17.2 ± 0.2 | 1.11 | 13.6 | 65 |
| p-SWNTs | 6.1 ± 0.2 | 0.40 | 15.1 | 166 |
| US-tubes | 0.63 ± 0.2 | 0.04 | 94.1 | 230 |

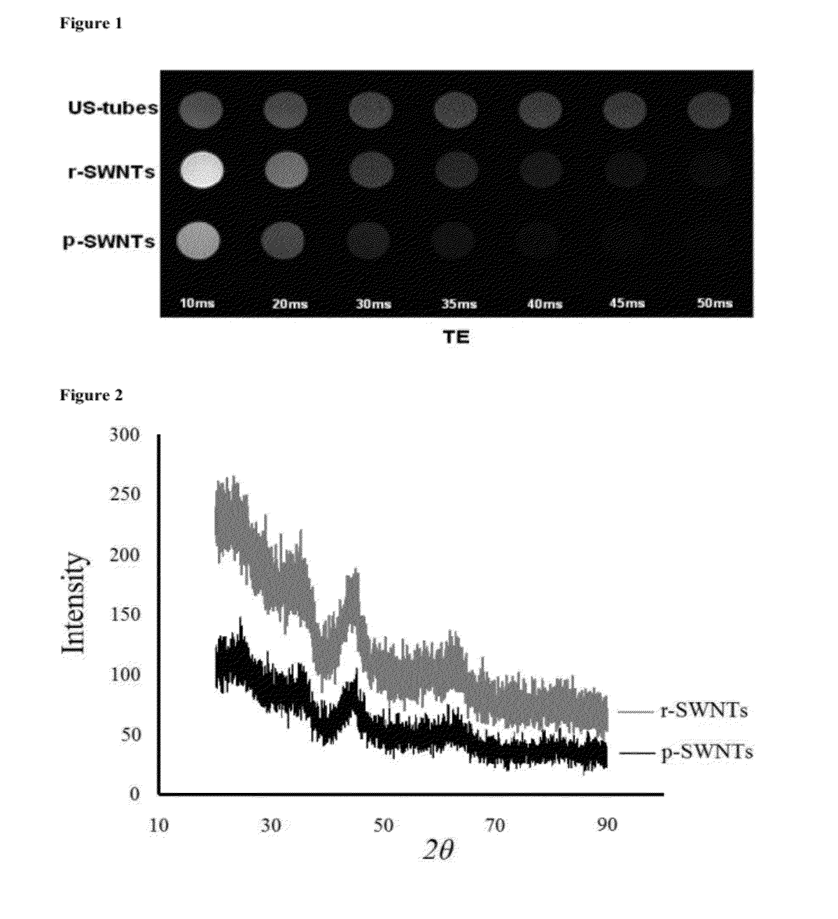

times, on the order of a few milliseconds. short T₂-relaxation times, on the order of a few milliseconds. The T₂-weighted MR phantom images of the SWNT solutions at different TEs are presented in FIG. 1. As the TE is increased, the SWNT materials lose their image contrast, which is a characteristic of high-performance T₂-weighted MR contrast agents.

The efficacy (relaxivity) of a contrast agent is expressed as a function of their concentration and T_{1,2} (MM₋₁s₋₁). Relaxivity is calculated using the relationship r₂=(R₂−R₀)/[CA], where R₂ and R₀ are the T₂-relaxation times (s⁻¹) of the sample and the dispersion medium, respectively, and [CA] is the concentration of the contrast agent expressed in mM. T₂-weighted MRI properties for carbon nanotube materials have been previously noticed and a separate study has shown that iron oxide-HiP_{CO} SWNT complexes can act as bimodal imaging agents (NIR fluorescence/MRI) where the MRI activity was attributed to the presence of small particles of iron oxide (SPIO). Assuming the proton relaxation effects of the SWNT materials used in this study are also derived from catalyst iron particles used in the growth process of HiPCO SWNTs, the relaxivities of the SWNT materials are calculated in Table 1. These results indicate that a high concentration of iron (or iron oxide) nanoparticles is not needed for optimal performance, since the p-SWNTs are an equally effective T₂-shortening agent in spite of having three times less iron than r-SWNTs. Furthermore, the US-tubes, which have the smallest iron content by far, possess the highest relaxivity. This unusual T2-relaxation behavior could be due to a different nature of the metal particles present in each of the SWNT samples since the samples undergo considerable modification during their purification (p-SWNTs) and cutting (US-tubes).

In order to better understand the T₂-relaxation properties, Xray diffraction (XRD) studies were performed on all the three SWNT samples. The US-tubes did not show any observable XRD peaks, which undoubtedly is due to their low iron content (<1%) below the detection limits of the instrument. The XRD data for the r-SWNT and p-SWNT samples are given in FIG. 2. As shown in the figure, both samples display similar XRD patterns. The peaks shown were assigned using JADE software, and the best fit was observed for Fe₃O₄. The XRD results demonstrate that the r-SWNT and p-SWNT materials are similar with respect to the iron particles present and that these iron particles are predominantly Fe₃O₄. Fe₃O₄ particles can act both as a ferromagnetic and superparamagnetic material depending on particle size.

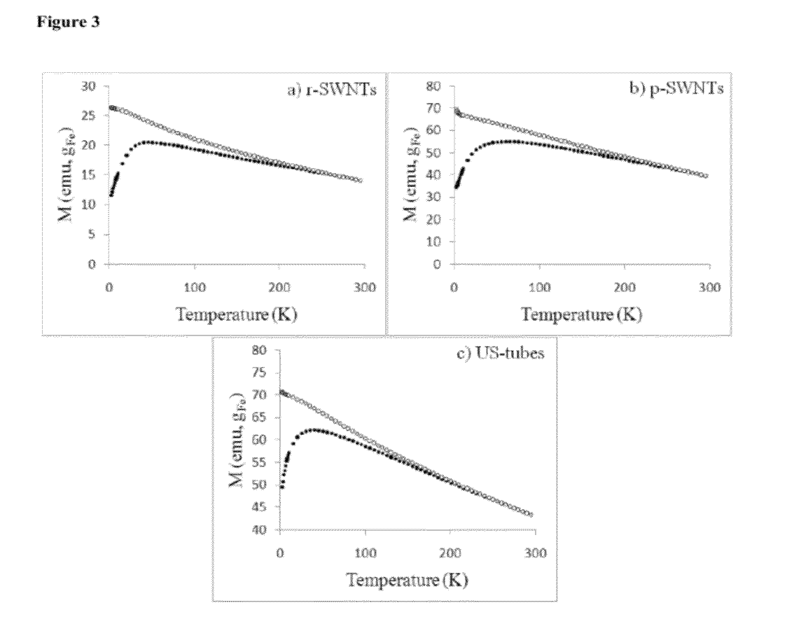

Since all the three SWNT materials are surprisingly effective T₂-agents, their magnetization properties were determined by SQUID magnetometry (FIG. 3). The r-SWNTs (FIG. 3a), p-SWNTs (FIG. 3b) and US-tubes (FIG. 3c) are all consistent with superparamagnetism: the zero-field-cooled (ZFC) curves are characterized by a mean blocking temperature T_{B}, above which the material is superparamagnetic and below which magnetic viscosity gives rise to a hysteretic magnetization loop. This cusp is a uniquely characteristic signature of either superparamagnetic or spin-glass states; however, all of our samples lacked the irreversibility of the field-cooled (FC) curve, or thermoremnant (TRM) magnetization which is characteristic of a spin-glass state. Furthermore, the maximum blocking temperature T_{B,max}, or the temperature where initial bifurcation between the ZFC and FC curves occurs, decreases with purification from r-SWNTs to US-tubes (FIG. 3). This indicates that as T_{B,max} approaches T_{B}, the net distribution of superparamagnetic domain sizes become more uniform. Interestingly, the US-tube sample shows the greatest relaxivity, though its magnetic susceptibility is far less than that of the r-SWNT and p-SWNT samples. This can be attributed to the smaller quantity, as well as the smaller size, of the iron particles in the US-tube samples, and also to the possible different nature of the iron particles present in the US-tubes (since characterization of these particles is not yet established). The advantage in relaxivity for the US-tubes over the other SWNT materials (a 1.5 fold advantage over p-SWNTs and a 4 fold advantage over r-SWNTs) when normalized for iron particle concentration (Table 1) suggests that the role of the carbon SWNTs themselves should not be ignored when interpreting their resultant magnetic and T₂-relaxation properties. In fact, theoretical studies have shown that finite zigzag carbon nanotube materials may be inherently paramagnetic by virtue of their chirality, diameter, and length, with shorter length tubes being potentially more magnetic. In addition the presence of defect sites (much more abundant in US-tubes than r-SWNTs or p-SWNTs) could produce enhanced magnetic properties in the nanotube materials as well. If the iron particles (as Fe3O4) alone were responsible for the T2-relaxation behavior in Table 1, a reasonably constant relaxivity might be expected, since the X-ray diffraction and magnetic data did not detect a significant difference in the nature of the iron particles or their magnetic behavior in the r-SWNTs and p-SWNT samples. Using a T_{B} of 40 K and the magnetic anisotropy constant K of bulk Fe₃O₄, 4×10⁵ erg cm⁻³ (the majority of residual catalyst in raw HiP_{CO} SWNTs exists as magnetite), we calculate a mean particle volume V=3.45×10⁻¹⁹ cm³, or a mean particle radius r=3.45 nm using T_{B}=KV/25 k_{B}. When compared to similarlysized iron oxide particles (2-4 nm) with a T₂-relaxivity of 72 mM⁻¹s⁻¹, the p-SWNTs and US-tubes far outperform their solely iron oxide-based counterpart. This fact suggests that superparamagnetic SWNT materials may be a distinct new class of T₂-weighted MRI contrast agent with performance components from both the iron oxide and the carbon SWNT material itself. The superior relaxivity of the p-SWNTs and US-tubes over the r-SWNTs is also noteworthy since these materials, when used as in vivo MRI agents, should demonstrate reduced metal-mediated toxicity.

The US-tubes, with their shorter length, superior relaxivity, and negligible metal content may well be the most promising SWNT material of all for in vivo MRI and magnetic cell labeling/trafficking studies. We are presently exploring this possibility for both empty US-tubes and Gd³⁺-ion-filled US-tubes (gadonanotubes) which are concomitantly highperformance T₁-weighted and T₂-weighted agents.

### Example 3

In this example, J774 mouse macrophages cell line (5×10⁵ cells/well) was maintained in DMEM culture media with 10% FBS and 1% S/P in a 6-well plate to allow cell adhering. The cells were then co-cultured with gadonanotubes solution, at a final concentration of (27.25 μM Gd, 185.5 mg C/L) for 24 hours. Labeled cells were then re-suspended in equal volume of 2× culture media and 2% agarose gel after washing with PBS, and transferred to lcc syringe (3.5 cm long) for MRI measurements. Five different concentrations of labeled cells phantoms (2.3×10⁶, 1.15×10⁶, 0.75×10⁶, 0.57×10⁶, and 5,500 cells/ml) were prepared. Another four phantoms with unlabeled cells (2.3×10⁶, 1.50×10⁶, 0.75×10⁶, 0.57×10⁶ cells/ml) were also prepared as controls. The mean cellular uptake of gadonanotubes was quantified using Inductively-coupled Plasma (ICP) analysis. Cytotoxicity of the gadonanotubes was tested in a 96-well plate (triplet) at various gadonanotubes concentrations (n=4) using MTS assay prior to experiment.

MRI was performed at a 3 T system (General Electric Milwaukee, Wis.) using a 35 mm I.D. research quadrature coil for relaxivities measurement and at a 9.4 T system (Bruker Biospec 94/20 USR) for cell visualization. For 3 T, R₂ and R₂* measurements of the phantoms were acquired with spin echo and gradient echo sequences (TR=1500 ms, TE=15, 30, 45, 60, 75, 100, 125 ms, FOV=5.0 cm, matrix=128×128, NEX=1, thickness=1 mm) respectively. Circular ROIs were drawn and the R₂ and R₂* were computed based on the mean intensity of each ROI in the phantoms. ΔR₂ and ΔR₂* of the labeled cell phantoms were calculated by subtracting the R₂ and R₂* values with that of the unlabeled cell phantoms controls. Sparsely distributed labeled cell phantom (5,500 cells/ml) was imaged at 9.4 T with a 3D spoiled gradient echo sequence (TR/TE=3000/40 ms, alpha=28.6°, FOV=0.64×2.56 cm, resolution=50 μm isotropic, NEX=12). Reconstruction was done using susceptibility weighted imaging (SWI) method to enhance the contrast effect for better visualization.

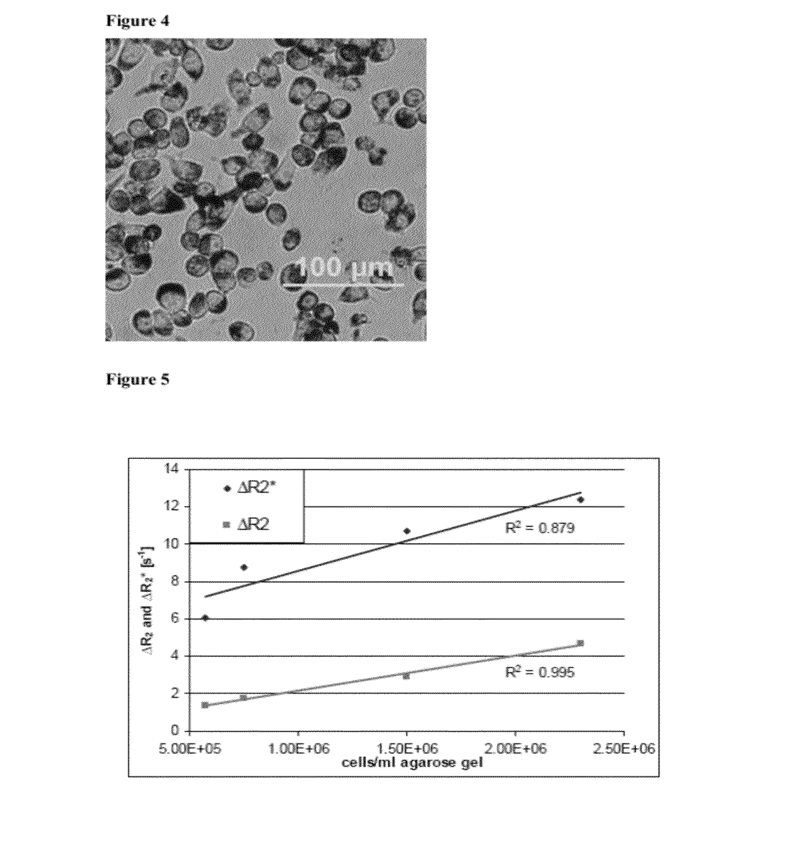

FIG. 4 shows the microscopic image of gadonanotubes labeled J774 cells. The cells appear black in the intracellular space, showing efficient gadonanotubes internalization. MTS assay shows no significant cytoxicity effect at the concentration of gadonanotubes used. ICP results show an average cellular uptake of 0.44±0.09 pg Gd/cell, corresponding to an uptake of ˜19.3±3.8 pg C/cell. FIG. 5 shows ΔR₂ and ΔR₂* measurements of the gel phantoms with gadonanotubes labeled cells at different concentration at 3 T. The ΔR₂ and ΔR₂* values increase linearly with an increasing number of labeled cells. This matches well to the ICP results showing increasing concentration of gadonanotubes (reflected as Gd³⁺ ions) in the phantoms, as shown in Table 3.

TABLE 3

| cells/ml | [Gd] μg/ml |
|---|---|
| 2.30 × 10⁶ | 0.85 |
| 1.50 × 10⁶ | 0.61 |
| 0.75 × 10⁶ | 0.43 |
| 0.57 × 10⁶ | 0.05 |

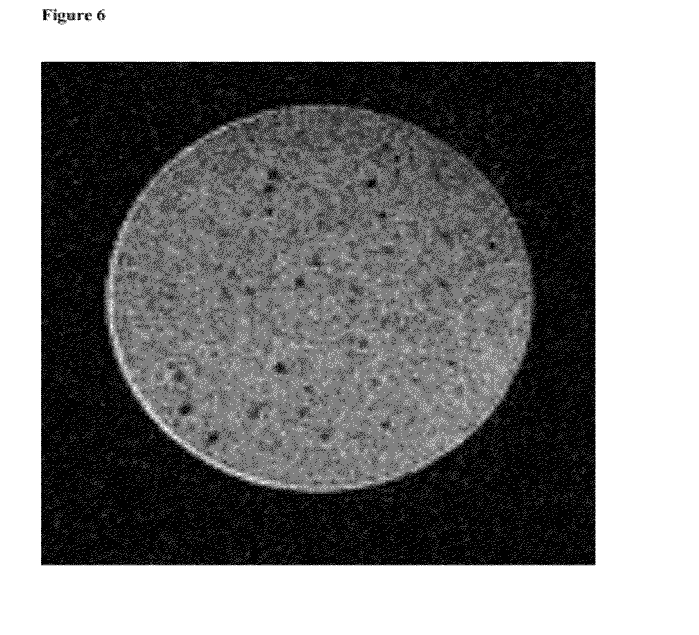

FIG. 6 shows a 9.4 T T₂*-weighted image of sparsely distributed cells (5,500 cells/ml) in agarose gel within a lcc syringe showing single cell visualization.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A contrast agent composition comprising: at least one carbon nanotube; and a metal catalyst.

2. The contrast agent composition of claim 1 wherein the at least one carbon nanotube is a single-walled carbon nanotube.

3. The contrast agent composition of claim 1 wherein the at least one carbon nanotube is an ultra-short carbon nanotube.

4. The contrast agent composition of claim 1 wherein the at least one carbon nanotube is formed by electric arc discharge.

5. The contrast agent composition of claim 1 wherein the at least one carbon nanotube is formed by high pressure carbon monoxide conversion.

6. The contrast agent composition of claim 1 wherein the metal catalyst is selected from the group consisting of: Fe, Fe₂O₃, Y/Ni, and Y₂O₃/NiO.

7. The contrast agent composition of claim 1 further comprising at least one member selected from the group consisting of a multimodal imaging agent and a drug.

8. The contrast agent composition of claim 7 wherein the at least one member is contained substantially within the interior of the at least one carbon nanotube.

9. The contrast agent composition of claim 1 further comprising at least one member selected from the group consisting of: a peptide, an antibody, a chemotherapeutic agent, and a radiotherapeutic agent.

10. The contrast agent composition of claim 9 wherein the at least one member is attached to the exterior wall of the carbon nanotube.

11. The contrast agent composition of claim 1 wherein the metal catalyst is present in an amount equal to or less than about 2% by weight of the composition.

12. The contrast agent composition of claim 1 wherein the metal catalyst is present in an amount of from about 0.5% to about 2% by weight of the composition.

13. A contrast agent composition consisting essentially of: at least one carbon nanotube; and a metal catalyst.

14. The contrast agent composition of claim 13 wherein the at least one carbon nanotube is a single-walled carbon nanotube.

15. The contrast agent composition of claim 13 wherein the at least one carbon nanotube is an ultra-short carbon nanotube.

16. The contrast agent composition of claim 13 wherein the metal catalyst is selected from the group consisting of: Fe, Fe₂O₃, Y/Ni, and Y₂O₃/NiO.

17. The contrast agent composition of claim 13 wherein the metal catalyst is present in an amount equal to or less than about 2% by weight of the composition.

18. The contrast agent composition of claim 13 wherein the metal catalyst is present in an amount equal to or less than about 2% by weight of the composition.

19. The contrast agent composition of claim 13 wherein the metal catalyst is present in an amount of from about 0.5% to about 2% by weight of the composition.

20. A method for obtaining a magnetic resonance image, the method comprising:
administering to a subject a contrast agent composition, wherein a contrast agent composition comprises at least one carbon nanotube and a metal catalyst; and
obtaining a magnetic resonance image of at least a portion of the subject in which the contrast agent is disposed.

21. The method of claim 20 wherein at least one carbon nanotube is a single-walled carbon nanotube.

22. The method of claim 20 wherein the at least one carbon nanotube is an ultra-short carbon nanotube.

23. The method of claim 20 wherein the metal catalyst is selected from the group consisting of: Fe, Fe₂O₃, Y/Ni, and Y₂O₃/NiO.

24. The method of claim 20 wherein the metal catalyst is present in an amount equal to or less than about 2% by weight of the composition.

25. The method of claim 20 wherein the metal catalyst is present in an amount of from about 0.5% to about 2% by weight of the composition.
